(19) United States
(12) Patent Application Publication
(10) Pub. No.: US 2017/0246626 A1
(43) Pub. Date: Aug. 31, 2017

(54) TEST DEVICE AND METHOD

(71) Applicant: CLINICAL GENOMICS PTY LTD, North Ryde, New South Wales (AU)

(72) Inventor: Howard Milne CHANDLER, Avoca Beach, New South Wales (AU)

(73) Assignee: CLINICAL GENOMICS PTY LTD, North Ryde, New South Wales (AU)

(21) Appl. No.: 15/508,314

(22) PCT Filed: Aug. 31, 2015
(86) PCT No.: PCT/AU2015/050510

§ 371 Date: Mar. 2, 2017

Related U.S. Application Data
(60) Provisional application No. 62/044,804, filed on Sep. 2, 2014.

(51) Int. Cl. B01L 3/00; G01N 33/72; A61B 10/00; G01N 33/52

(52) U.S. Cl. CPC B01L 3/5023; G01N 33/52; G01N 33/726; G01N 33/725; A61B 10/0038; B01L 2200/0605; B01L 2200/141; B01L 2300/043; B01L 2300/0681; B01L 2300/069; B01L 2300/161

(57) ABSTRACT

Provided is a test device for testing an analyte in a collected sample comprising: an enclosure having at least one sample receiving port for receiving the sample; a test strip located within the enclosure, the test strip containing at least one reagent for detecting the analyte and for providing an indication showing a test result for the sample, the enclosure including a detection arrangement for allowing detection of the indication of the test strip; a sample receiving matrix positioned behind the at least one sample receiving port and having a defined saturation capacity, the sample receiving matrix being impregnated with reagents for pre-treatment of the sample and being in liquid conductive communication with the test strip. Also provided is a method for use of said test device comprising: delivering one or more samples to the at least one sample receiving port to saturate the sample receiving matrix such that a quantified amount of the sample is transferred to the test strip and an indication of the test result is effected.

### FIELD OF THE INVENTION

The present invention is generally directed to diagnostic and analytical systems for the detection of an analyte in a sample, and in particular to a test device and method applicable for this application. While the present invention will be described with respect to its application in screening for lower intestinal disorders, particularly in screening for colorectal cancer, it is to be appreciated that the invention is not restricted to this application, and that other applications are also envisaged.

### BACKGROUND TO THE INVENTION

Guaiac-based Faecal Occult Blood Tests (gFOBTs) and Faecal Immunochemical Tests (FITs) are used to screen for the presence of blood in the stool as a possible indicator of colorectal cancer (CRC), or its precursor lesions. While proven effective, compliance with these screening tests is typically less than 50%, generally believed due to an aversion of faecal handling. Typically stool must be collected in a sling and several samples collected using a stick or spear. The stool sample may be resuspended in a liquid reagent prior to sending to a Pathology Lab, where various manipulations and reagent additions are required to achieve a test result. Other negative aspects of these tests include that significant degradation of the sample may occur in transit and the laboratory development may be costly in space, labour, time and/or the requirement for expensive equipment for their automated processing and reading.

U.S. Pat. Nos. 7,972,871 and 8,389,287 respectively describe a testing device and collection method that addresses in part some of the above issues by simplifying the testing and collection processes. U.S. Pat. No. 8,389,287 describes a collection process utilising a brush or brush-like device to sample toilet bowl water from on or around the stool after defaecation. The brush can then be used to transfer the sample to a sample collection device, where it dries, thereby ensuring analyte stability during transport. U.S. Pat. No. 7,972,871 describes the conversion of the sample collection device into a test device by inserting an immunochemical test strip into the sample collection card. Reagent addition to the collection card mobilises the sample and transports it to the test strip, where anti-human haemoglobin antibodies label and immobilise any haemoglobin in the sample thereby indicating the presence or absence of faecal occult blood in the sample.

All currently described FOBTs and FITs require faecal samples to be sent to a pathology lab for testing, with inherent delays between sample collection and testing of the sample. This can result in sample degradation, an issue that is particularly important in relation to FOBTs and FITs due to the relatively rapid deterioration of the haemoglobin molecule over time.

Furthermore, a number of steps (namely sample collection, collection device delivery and testing) are required before a test result can be obtained for the sample. It is therefore preferable to minimise the number of steps involved before the test result is obtained for both practical and commercial reasons.

Where large numbers of tests need to be processed, for example in population screening programmes, it is currently necessary to automate the test development procedures to ensure that the tests are processed within an appropriate period of time. A substantial and financial investment is therefore involved in setting up and running such automated systems and having the necessary skilled personnel to manage the automated systems and process the results.

It is an object of the present invention to address one or more of the above mentioned disadvantages of the existing test systems and methods.

### SUMMARY OF THE INVENTION

According to one aspect of the present invention, there is provided a method for testing an analyte in a collected sample using a test device including an enclosure having at least one sample receiving port for receiving the collected sample; a test strip located within the enclosure, the test strip containing at least one reagent for detecting the analyte and for providing an indication showing a test result for the collected sample, the enclosure including a detection arrangement for allowing detection of the indication of the test strip; and a sample receiving matrix positioned behind the at least one sample receiving port and having a defined saturation capacity, the sample receiving matrix being impregnated with reagents for pre-treatment of the sample and being in liquid-conductive communication with the test strip, said method comprising: delivering one or more samples to the at least one sample collection port to saturate the sample receiving matrix such that a quantified amount of the sample is transferred to the test strip and an indication of the test result is effected.

As used herein, the term “liquid-conductive communication” shall be taken to mean that a sample collected within a solvent such as water, when applied via the sample collection port to the sample receiving matrix is capable of being in liquid-conductive communication with the test strip under sufficient conditions of hydration to enable transfer of at least part of said sample, or a component thereof, to the test strip.

The test strip may be formed from an elongate strip of hydrophilic material. One end of the strip may provide a first location for sample addition (referred to as a sample receiving port), the opposing end of the strip providing a second location for the indication, such as a visual indication, showing the test result. The first location of the test strip may therefore be located in liquid conductive communication with the sample receiving port. The transferred sample may then migrate along the test strip to a downstream area of the test strip where the presence of the analyte may be indicated.

The test device may be an extension of the test strip described in U.S. Pat. No 7,972,871. The sample receiving matrix may be added onto the end of the test strip and laminated as a single web, ensuring direct contact between the sample receiving matrix and the test strip. This web may then be impregnated with the same solutions used to prepare the sample receiving matrix and test strip. In addition, the distance between the sample receiving matrix and the indication zone, for example a visual indication zone such as a gold conjugate zone, may be varied, to ensure adequate mixing and/or rehydration of the collected sample with the dried buffer/reagents on the sample receiving matrix.

In applications where the present invention is used for detection of faecal occult blood, the test strip may include, for example, an area with dried down gold-conjugated polyclonal anti-human haemoglobin (Hb) antibody downstream from the sample application area. A line of immobilised monoclonal anti-Hb antibody may be located further downstream on the test strip. This line captures and accumulates any gold-conjugated Ab-Hb complexes that are produced if Hb is present in the sample, to thereby produce a visible line. This line therefore provides visual confirmation of the presence of Hb in the water sample. Alternatively, other disclosing agents may be used which enable the indication to be detected, for example fluorescent dyes or particles, or magnetic particles. In these cases the detection of bound aggregates of Hb and the disclosing agent may be by other means, e.g. spectrometry, fluorimetry or magnetometry. This can be a convenient alternative means of preventing a patient from being able to read their own test results. Accordingly, reference to an “indication” should be understood as a reference to both an indication which is visible to the naked eye and one which requires additional means to enable its detection, such as via spectrometry, fluorimetry or magnetometry.

Most immunochemical tests suffer from the prozone phenomenon, which causes a diminished signal at very high analyte concentrations. Thus, there is a risk that advanced cancers, where there is heavy bleeding, may provide a weak or borderline result that may be missed. The test strip of the present invention may therefore further include reagents which detect the pseudoperoxidase activity of haem such as a peroxidase reagent and a chromogen reagent, preferably at a distal end of the test strip and beyond the immunodetection zone, that allows for the detection of high haemoglobin (Hb) levels by providing a prominent colour reaction in the presence of haem. Without limiting the present invention in any way, the pseudo peroxidase reaction detects the stable haem of the haemoglobin and the immunochemical reaction detects the labile globin protein. By combining an immunological test for globin with a non-immunological test for haem, the incidence of false negative results occurring due to the prozone phenomenon are minimised. The use of a two stage testing procedure directed to testing for both the haem and the globin components of haemoglobin also permits differentiation of upper gastrointestinal tract bleeding from lower gastrointestinal tract bleeding. Since the globin protein of haemoglobin does not survive passage through the upper gastrointestinal tract, a positive result for globin therefore indicates lower gastrointestinal tract bleeding.

The reading for haem therefore supplements the immunochemical reading and makes frank bleeding immediately obvious, thereby eliminating the risk of missing an advanced cancer due to prozone-related false negatives which can occur where high concentrations of blood are present in the patient sample.

A test strip suitable for the present invention is described in U.S. Pat. No. 7,972,871, details of which are incorporated herein by reference.

The sample receiving matrix positioned behind the sample receiving port facilitates communication of the collected sample with the test strip located within the enclosure.

The sample receiving matrix may be formed from a hydrophilic material that can absorb at least part of the collected sample delivered to the sample receiving port.

The sample receiving matrix may be dimensioned to provide a volume sufficient to receive one or more samples and to release a quantified amount of the collected sample to the test strip to complete the test.

The sample receiving matrix may also provide filtration of any of the solids, for example faecal solids, from the collected sample before transfer to the test strip.

The sample receiving matrix may preferably contain one or more reagents to solubilise and buffer any analyte(s) in the sample before migrating to the test strip.

The sample receiving matrix may also or alternatively contain one or more lytic agents to lyse and release the contents of any cells present in the collected sample.

The sample receiving matrix may also or alternatively contain one or more surfactants to prevent non-specific binding and loss of analyte to the matrix and test strip.

The sample receiving matrix may also or alternatively accommodate a disclosing reagent such as, for example, an anti-globin antibody conjugated to colloidal gold. As described previously, this reagent may be immobilised on the test strip. However, relocation of the reagent to the sample receiving matrix may enable longer and more uniform mixing of the sample and reagent, thereby improving reaction kinetics and potentially improving the test performance.

The sample may be collected by dipping at least a portion of the test device into a liquid containing the sample such that the sample receiving matrix comes into contact with the liquid to thereby collect said sample. In this arrangement, the sample receiving port would be located on the portion of the test device being dipped. The test device may be dipped one or more times into the liquid for collecting one or more samples. The defined saturation capacity of the sample receiving matrix enables the appropriate volume of liquid to be retained by the test strip.

The liquid containing the sample may alternatively be collected using a sample collection apparatus for collecting the sample and delivering the sample to the sample receiving port. The sample collection apparatus may collect one or more samples and may deliver one or more samples to the sample receiving port.

In FOBT and FIT applications, the liquid containing the sample may be the toilet water within which the person being tested has deposited faeces. The toilet water can therefore act as both the sample, and the test developer.

The sample collection apparatus used to collect the sample may preferably be in the form of a brush or brush like apparatus having flexible or semi-flexible bristles. The advantage of using a brush or brush like apparatus is that it allows the collection of liquid samples within the bristles of the brush or brush-like apparatus. Such a sample collection apparatus is particularly applicable for use in FOBTs and FITs because it allows the collection of faecal material released from a stool located within the water of a toilet bowl. In addition the bristles allow brushing around the stool to disperse any blood into the surrounding water. Furthermore, the water collected within the bristles of the brush or brush-like apparatus can act as a solvent to facilitate the liquid-conductive communication of the collected sample within the test strip. The brush or brush-like apparatus can be specified to collect a sufficient volume of liquid to complete the test. The brush or brush-like apparatus may have a liquid holding capacity equal to or greater than the defined saturation capacity of the sample receiving matrix. This ensures that the sample delivered by the brush or brush-like apparatus saturates the sample receiving matrix. In an alternative embodiment, and as discussed further below, the brush may have a liquid holding capacity less than than the defined saturation capacity of the sample receiving matrix.

The brush may be of any suitable size. Where a large brush with capacity to deliver larger volumes of liquid is used, only one application of the liquid sample may be required. However in one embodiment, in order to enable the use and packaging of a smaller brush, it may be desirable to deliver two sequential liquid samples to the receiving port. This may also be desirable from the point of view that faecal contamination may cause an undesirable level of back ground discolouration to the test strip that may possibly obscure a borderline positive result. In order to minimize the possibility of this occurring, one may elect to use the following protocol:
(a) Use the brush to add a toilet water sample to the sample port;
(b) Flush the toilet; and
(c) Use the same brush to add a sample of clean water from the toilet bowl to the sample port.

In this embodiment the smaller brush or brush-like apparatus may have a liquid holding capacity less than the defined saturation capacity of the sample receiving matrix. It would be appreciated by the skilled person that the total volume of liquid delivered by the smaller brushes or brush-like apparatuses is sufficient to saturate the sample receiving matrix.

The advantages of using a brush or brush-like apparatus is described in more detail in U.S. Pat. No. 8,389,287, details of which are incorporated herein by reference.

The term “brush” herein is used to denote an apparatus comprising a stem or handle, usually elongate, and a clump, bunch or group of bristles, hair or other similar flexible or semi-flexible elongate strands, laminar flaps or the like attached to the stem or handles. The term “brush-like apparatus” is used herein to denote an apparatus which is similar to a brush in that it includes a bunch, clump or group of bristles, hair or other similar flexible or semi-flexible elongate strands, laminar flaps or the like. Whilst reference is made throughout the present specification to the collection of sample within the bristles of a brush or brush-like apparatus, it is to be understood that the reference to “bristles” is used to include the hairs or other similar flexible or semi-flexible elongate strands, laminar flaps or the like of a brush or brush-like apparatus.

The detection arrangement for allowing detection of the indication of the test strip may preferably include at least one port provided in the enclosure and located over the second location of the test strip that exhibits the indication. A visible indication could be viewed through this port or an indication not visible to the naked eye could be detected through this port, such as using fluorimetry. Alternative viewing means are however also envisaged. A transparent section may for example be provided for the enclosure to allow for the viewing a visual indication.

The visual indication may be for example, in the form of one or more lines extending across the exposed portion of the test strip. The position of the lines and/or the colour density of the or each line may provide the visual indication of the test result. A general assessment of the test results may be obtained by viewing the visual indication with the naked eye. Alternatively, a quantitative result may be obtained by using a viewing apparatus for measuring the colour density and distribution of the one or more lines of the visual indication.

At least one inspection port may also preferably be provided downstream of the sample receiving port to allow observation of a visual indication confirming the flow of the collected sample through the test strip. For example, a coloured dye may be used to indicate the mobilisation and flow of the collected sample through the test strip. The provision of the inspection port will allow the test subject to determine whether or not the collected sample has been properly transferred into the test strip, with a further sample addition being required if no flow is observed. In one embodiment, the inspection port is located at the distal end of the test strip, between the control line and the absorbent. A band of water soluble dye (such as food dye) is dried onto this region of the test strip during its manufacture. The sample is applied to the receiving port (for example using a brush or by dipping the device into a liquid sample). The liquid sample is contacted with the sample port for a period of time until the band of dye is seen to be mobilised through the inspection port. Thereafter the contact can be ended. Where a brush is used, this will typically take 15-20 seconds of contacting the brush with the receiving port. If no flow is observed within 30 seconds, then another delivery of sample is required.

The enclosure may be preferably formed from a sheet of material that may be folded to form adjacent panels of the enclosure. The sample receiving, viewing or detection ports may be located in at least one of the enclosure panels, for example, a front panel. The test strip may be secured to at least one of the enclosure panels, for example, a rear panel. The sheet material that could be used may include plastic or waterproof cardboard.

An obscuring arrangement may be provided to at least hide the indication. This is likely to be of particular significance, for example, where the indication is a visual indication and it is desired that the patient not be able to read the test result. It may also be useful if the detection means, even if not visible to the naked eye, is preferably not exposed to daylight, such as a fluorescent marker. This arrangement may include the enclosure including at least one hinged flap for covering at least one of said ports. Preferably two said hinged flaps may be provided, one flap being locatable over the sample receiving port, the other flap being locatable over the viewing port. The flap(s) may be formed from the same sheet of material as the rest of the enclosure. The provision of a said flap over the sample receiving port provides sample containment and therefore also saves the test subject from the embarrassment of providing an exposed sample. Furthermore, said flap over the sample receiving port provides for hygiene as it seals the port and prevents faecal contamination. The provision of a said flap over the viewing port limits any anxiety that may arise from the test subject observing the visual indication provided by the test.

An identifier means may preferably be provided on the enclosure that links the test subject to the test results. This visual indicator may preferably be in the form of a coded label that may be used to seal the flap over the sample port. The test result and the coded ID may then be captured in a digital image linking the test subject to the test result.

According to another aspect of the present invention, there is provided a test device for testing an analyte in a collected sample comprising: an enclosure having at least one sample receiving port for receiving the collected sample; a test strip located within the enclosure, the test strip containing at least one reagent for detecting the analyte and for providing an indication showing a test result for the collected sample, the enclosure including a detection arrangement for allowing detection of the indication of the test strip; a sample receiving matrix positioned behind the at least one sample receiving port and having a defined saturation capacity, the sample receiving matrix being impregnated with reagents for pre-treatment of the sample and being in liquid-conductive communication with the test strip, wherein one or more said samples can be delivered to the at least one sample receiving port to saturate the sample receiving matrix such that a quantified amount of the sample is transferred to the test strip and an indication of the test result is effected.

As previously described, the sample can be collected by dipping the portion of the test device where the sample receiving port is located into a sample containing liquid. Alternatively, the sample may be contacted by a sample collection apparatus.

The method and test device according to the present invention may preferably be used for detecting occult blood and/or other indicators of lower gastrointestinal disorders. This allows the method according to the present invention to be used for faecal occult blood tests and faecal immunochemical tests. The samples may preferably be toilet bowl water taken from the vicinity of a stool. The sample collection apparatus may be a brush or brush-like apparatus having flexible or semi-flexible bristles may be used to collect the sample. The sample may be collected within the bristles and may collect sufficient water as a solvent to complete the test. The volume of the sample collected may be less than, equal to or greater than the defined saturation capacity of the sample receiving matrix. Where the volume of the sample collected is less than the defined saturation capacity of the sample receiving matrix it is to be expected that two or more deliveries of sample will be applied to the sample receiving port in order to effect saturation of the sample receiving matrix.

The present invention provides significant advantages over known FOBT and FIT systems because it allows testing of a collected sample at the time of collection, that is, the indication becomes effected automatically and rapidly once the sample is delivered to the sample receiving port since the sample is immediately enabled to wick along the full length of the test strip. The result is stable and thereby enabled to be detected at a later point in time, such as after delivery to a pathology laboratory. This then avoids known sample degradation losses of existing FOBT and FIT systems that require the collected sample to be delivered to an off-site pathology lab. Furthermore, automated test completion systems used in pathology labs to conduct the test completion are not required where a visual indication is used. Furthermore, the self-testing aspect of the test device of the present invention makes it suitable for home and field use where no laboratory facilities are available.

Throughout this specification, unless the context requires otherwise, the word “comprise”, and/or variations such as “comprises” or “comprising”, will be understood to imply the inclusion of a stated integer or step or group of integers or steps but not the exclusion of any other integer or step or group of integers or steps.

### BRIEF DESCRIPTION OF THE DRAWINGS

It will be convenient to further describe the invention with respect to the accompanying drawings, which illustrate a preferred embodiment of the test device according to the present invention. Other embodiments of the invention are possible, and consequently, the particularity of the accompanying drawings is not to be understood as superseding the generality of the preceding description of the invention.

In the drawings:

FIG. 1 is a front view of a first embodiment of a test device according to the present invention showing the sample receiving port.

FIG. 2 is a front view of the test device of FIG. 1 showing the viewing port.

FIG. 3(a) is a front view of an embodiment of a test device of the invention showing the first and second flaps and inspection port. The cellphone indicates the relative size of the device.

FIG. 3(b) is a front view of the test device with the first flap open to reveal the sample addition port and sample receiving matrix.

FIGS. 4, 5 and 13 are plan views of three embodiments of the device of the present invention prior to final assembly.

FIG. 6 shows an immunochromatographic test strip according to one embodiment of the invention, including test strip, sample application (first) zone, second reagent (viewing) zone; first (disclosing) reagent (intermediate) zone, flow confirmation line of water-soluble dye.

FIG. 7 shows the inspection window in the outer panel of one embodiment of the invention, including second flap (covers the viewing port), inspection port, flow confirmation line of water-soluble dye.

FIG. 8 is a series of front views of a second embodiment of a test device according to the present invention showing the change in visual indication as a function of the test results,

FIGS. 9(a) and 9(b) are respectively an image showing the visual indication of the test device of FIGS. 4 and 5 being read using a visual detection reader and the resultant reading obtained by the reader of the visual indication,

FIGS. 10(a) and 10(b) are respectively a graphical representation of dose-response curves of the buffer system and stool system. Human haemoglobin was diluted at indicated concentrations and applied to the sample receiving port. Cards were read using a digital reader and signal intensities of control line and test line were expressed as ratio metric units.

FIG. 11(a) and (b) are respectively graphical representations of analyte stability at 25° C., and analyte stability at 40° C. in a buffer system. Human haemoglobin was diluted at indicated concentrations and applied to the sample receiving port. Cards were read using a digital reader and signal intensities of control line and test line were expressed as ratio metric units. Cards were stored at 25 degrees Celsius for 21 days and read at indicated intervals.

FIG. 12 is a product description table comparing the current commercially available quantitative fecal immunochemical test devices versus the device described herein

### DETAILED DESCRIPTION OF THE INVENTION

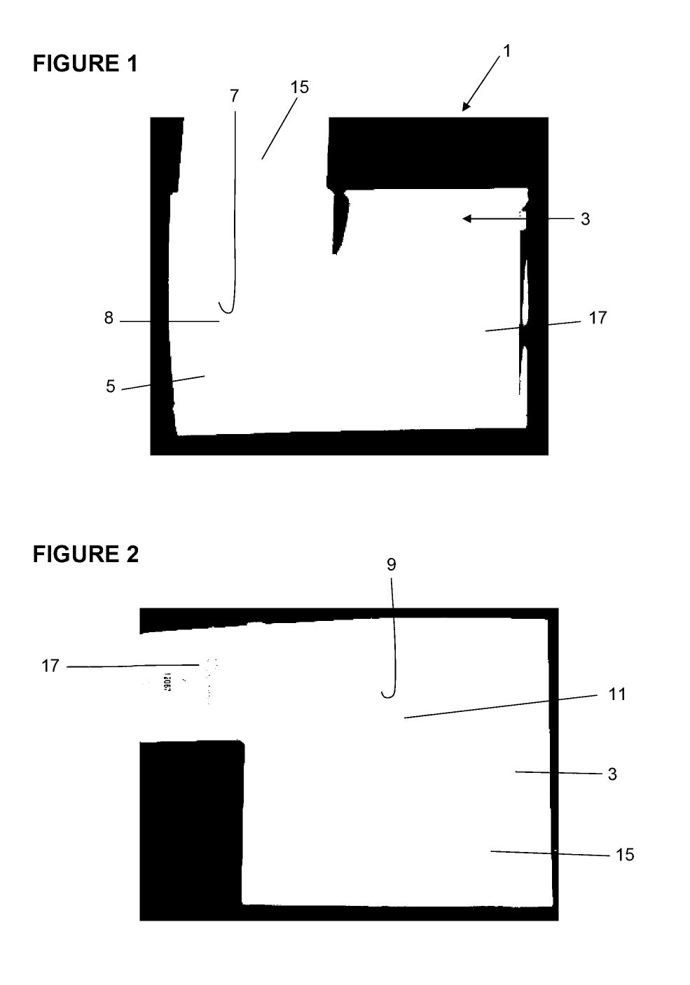

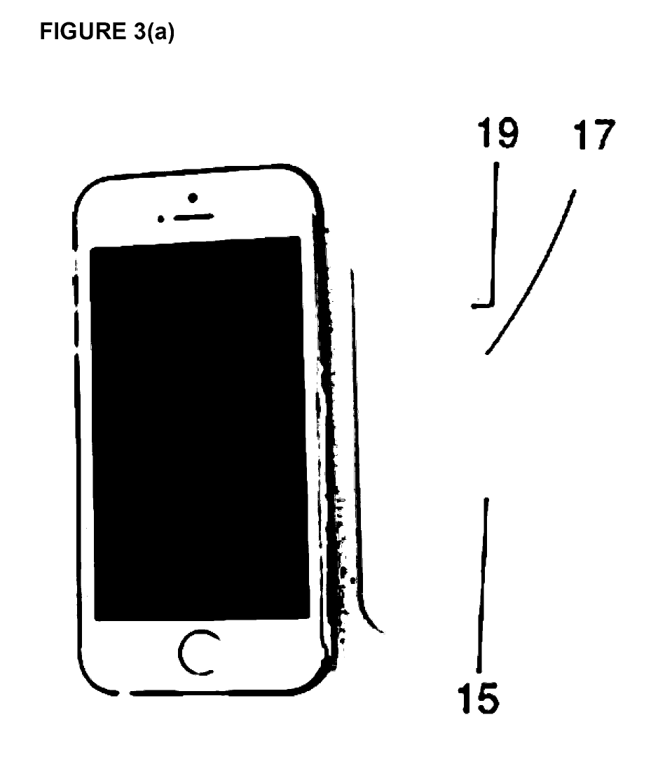

FIGS. 1 and 2 respectively show the assembled test device 1 according to the present invention. The test device 1 includes an enclosure 3 having a front panel 5. A sample receiving port 7 is provided on one side of the front panel 5 as shown in FIG. 1. A viewing port 9 is provided on the other side of the front panel 5 as shown in FIG. 2. A first flap 15 is provided to cover the sample receiving port 7, while a second flap 17 is provided to cover the viewing port 9.

A test strip 11 is accommodated within the housing 3, and a portion of the test strip 11 can be seen through the viewing port 9 in FIG. 2. The test strip 11 contains at least one reagent for detecting an analyte within a sample being tested by the test device 1.

A sample receiving matrix 8 is positioned behind the sample receiving port 7. The purpose of the sample receiving matrix 8 is to receive the sample and facilitate the transfer of a sample delivered to the sample receiving port 7 to the test strip 11. The sample receiving matrix 8 can contain one or more reagents to solubilise and buffer any analytes in the sample before migrating to the test strip 11. The matrix 8 may also or alternatively contain one or more surfactants and lytic agents to lyse and release the contents of any cells present in the collected sample.

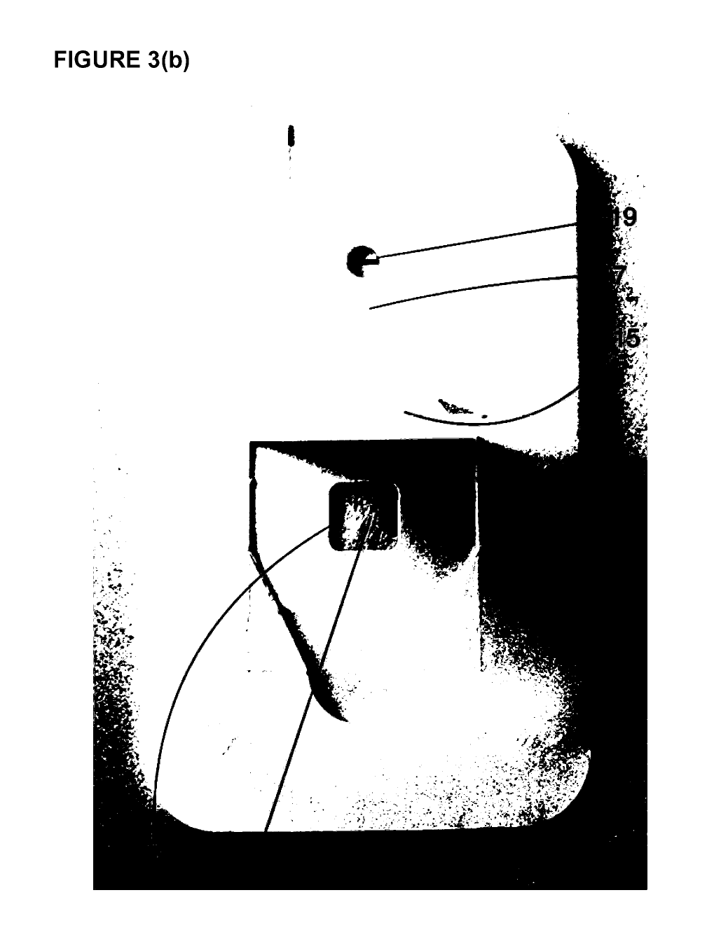

FIGS. 3(a) and 3(b) depict another embodiment of the device of the invention. FIG. 3(a) shows the first flap 15 covers the sample receiving port, second flap 17 covers the viewing port and inspection port 19. FIG. 3(b) shows the sample receiving port 7, sample receiving matrix 8, first flap 15, second flap 17, inspection port 19.

The collection apparatus for collecting the sample may vary depending on the application in which the tested device 1 is being used. In the case of FOBTs and FITs, the collection apparatus may be provided by a brush or brush like apparatus (not shown) having flexible or semi flexible bristles. The advantage of using such a brush as a collection apparatus is that it allows for the collection of a sample from the vicinity of a stool located within the water of a toilet bowl, the brush further collecting some of the water within the bristles of the brush. This water can subsequently act as a solvent to facilitate the liquid-conductive communication of the collected sample with the test strip 11. The brush or brush-like apparatus can also collect sufficient water to complete the test.

The sample is delivered to the sample receiving port 7. Any faecal solids within the sample are then filtered by the sample receiving matrix 8 before the remaining part of the sample is transferred to the test strip 1 by virtue of the liquid conductive communication with the test strip 11. The collected sample then migrates along the test strip 11 to areas within the strip containing the reagents (25 & 23) for detecting the analyte within the sample. In the case of FOBTs and FITs, the analyte will be haemoglobin. The test strip 11 furthermore provides a visual indication of the test result (23) that can be viewed through the viewing port 9.

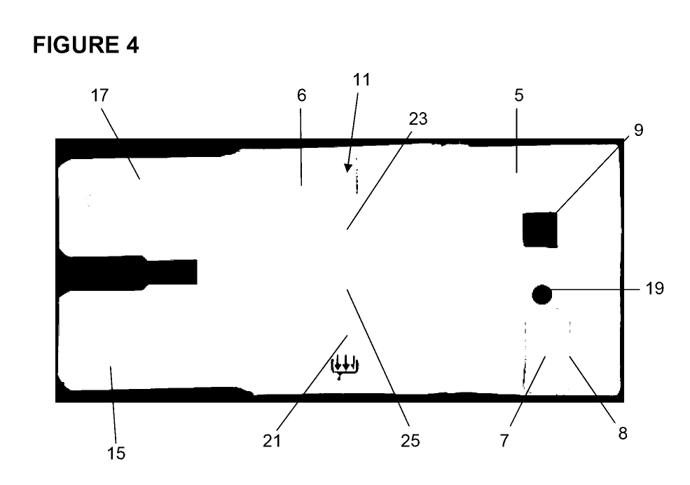

FIG. 4 shows test device 1 prior to final assembly showing in more detail the various components of the test device 1. The housing 3 includes a rear panel 6, with the test strip 11 being secured to that rear panel 6. Attached to one edge of the rear panel 6 is the front panel 5 through which are respectively located the sample receiving port 7 and the viewing port 9. An inspection port 19 (which is not shown in FIGS. 1 and 2) is also optionally provided on the front panel 5 between the sample receiving port 7 and the viewing port 9. The purpose of the inspection port 19 is to allow a visual inspection of the transfer of the sample along the test strip, for example by the inclusion of a coloured dye within the test strip showing sample transfer along the test strip 11. The two flaps 15, 17 are attached to the opposite edge of the rear panel 6 from the front panel 5. The sample receiving matrix 8 is shown in FIG. 4 to be significantly larger in dimension than the area of the sample receiving port 7. The matrix 8 is so dimensioned to provide a sufficient volume to absorb one or more collected samples, and in particular, a defined saturation capacity equal to the volume of the sample required to complete the test of the test device 1 to thereby standardise an amount of the sample used in the test.

The test device 1 is assembled by folding the front panel 5 over the rear panel 6 thereby covering the test strip 11. The sample receiving part 7 is then located over a first location 21 on the test strip 11 where the sample is initially transferred from the sample receiving matrix 8. The viewing port 9 is located over a second location 23 on the test strip 11 where the visual indication showing the test result is shown. The inspection port 19 is located at an intermediate location 25 of the test strip 11 between the first and second locations 21, 23.

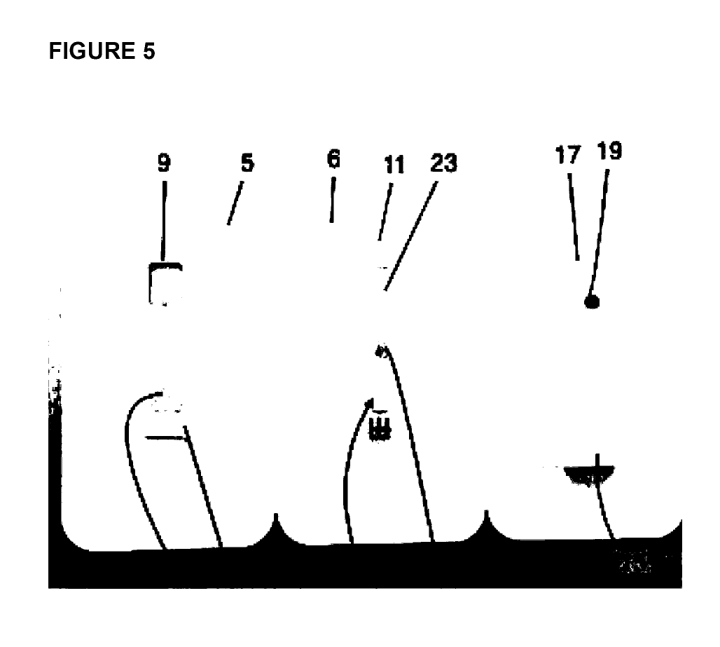

FIG. 5 is another embodiment of the test device and depicts front panel 5, rear panel 6, sample receiving port 7, sample receiving matrix 8, viewing port 9, test strip 11, first flap 15 (covers the sample receiving port), second flap 17 (covers the viewing port), inspection port 19, test strip first location 21 (where sample is transferred from the receiving matrix), second zone 23 (detection zone), intermediate location 25 (test strip disclosing reagent). Assembly: glue panel 5 over 6 and the right hand panel over 5.

The method used to run the test using the test device can include the following steps:
a) use a brush to collect a toilet water sample;
b) delivering the sample collected by the brush to the sample receiving port 7;
c) confirm the transfer of the sample along the test strip 11 by visual inspection of the flow confirmation line of dye 26 through the inspection port 19; and

d) view the visual indication provided by the test strip 11 through the viewing port 9.

The brush may be sized to collect a quantity of the toilet water sample to fully saturate the sample receiving matrix 8. Alternatively, a smaller brush may be used to deliver more than one sample to fully saturate the sample receiving matrix 8.

It is also possible that the faecal contamination may cause an undesirable level of background discolouration to the test strip 11 that may obscure a borderline positive test result. The above test method can therefore be altered by using a small brush to add a toilet water sample to the sample receiving port 19;

flushing the toilet; and

using the same brush to add a “chase” of clean water from the toilet bowl to the sample receiving port 7.

It is also envisaged that the sample could be collected from the toilet water by dipping the test device 1 directly into the toilet water thereby eliminating the need for the brush or other sample collection apparatus. The portion of the test device 1 having the sample receiving port 7 is dipped into the toilet water to thereby saturate the underlying sample receiving matrix 8 with the toilet water sample. The test results can then be obtained in the same manner by viewing the visual indication provided by the test strip 11 through the viewing port 9.

The visual indication provided by the test strip 11 may be in the form of one or more lines extending across the end of the test strip 11 viewable through the viewing port 9.

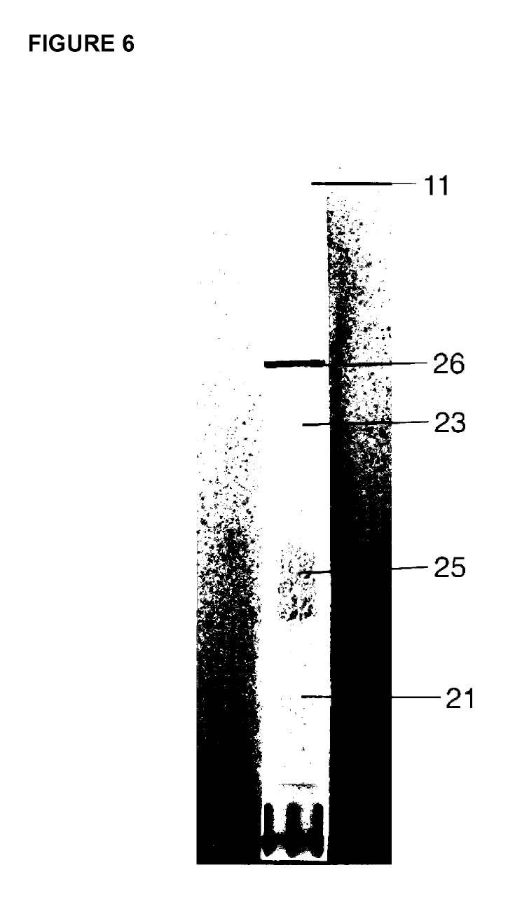

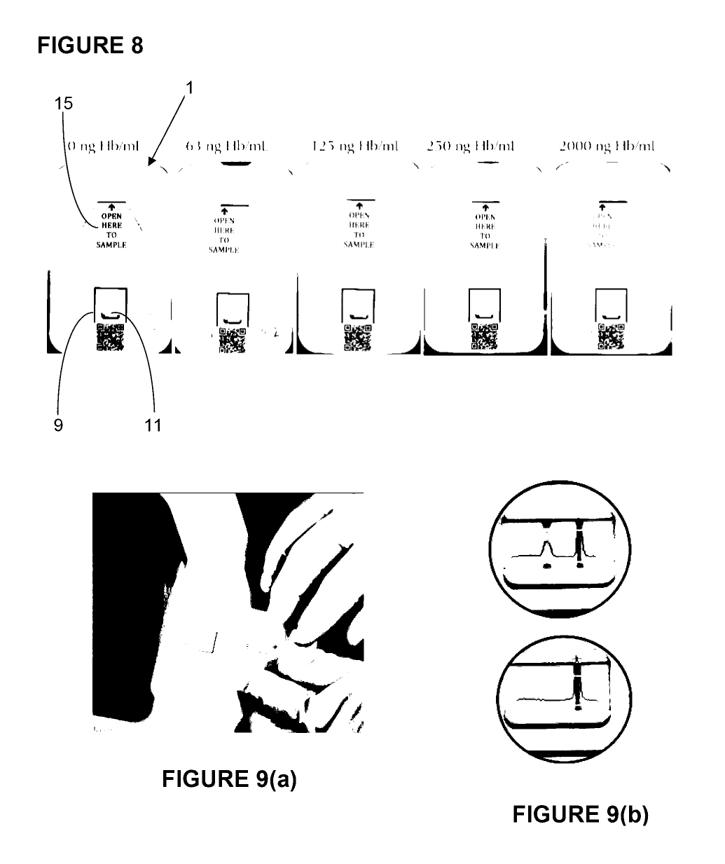

FIGS. 6, 7 and 8 show a series of images of other embodiments of the test device 1 according to the present invention. The same reference numerals are used for the same features as the first embodiment of the test device 1 for clarity reasons.

The visual indication may provide a quantitative result depending on the number of lines, and the colour density of the or each line. The visual indication for progressively increasing test values are respectively shown when looking from the left most sample to the right most sample of the image, shown in FIG. 6.

More particularly, FIG. 6 shows an immunochromatographic test strip according to one embodiment of the invention, including test strip (11), sample application (21) (first zone), second reagent (viewing) zone (23); first (disclosing) reagent (intermediate) zone (25), flow confirmation line of water-soluble dye (26).

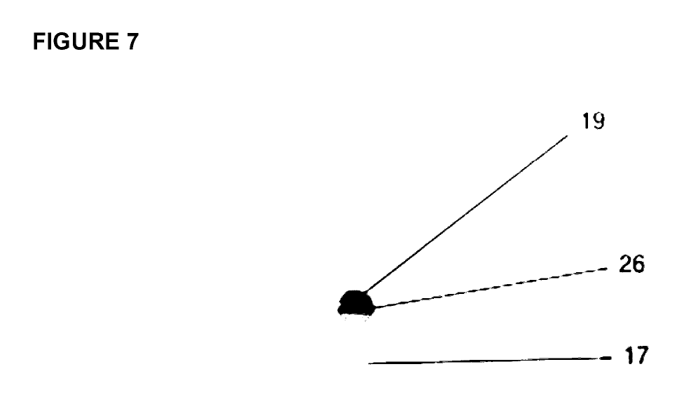

FIG. 7 shows the inspection window in the outer panel of one embodiment of the invention, including second flap (covers the viewing port) (17), inspection port (19), flow confirmation line of water-soluble dye (26).

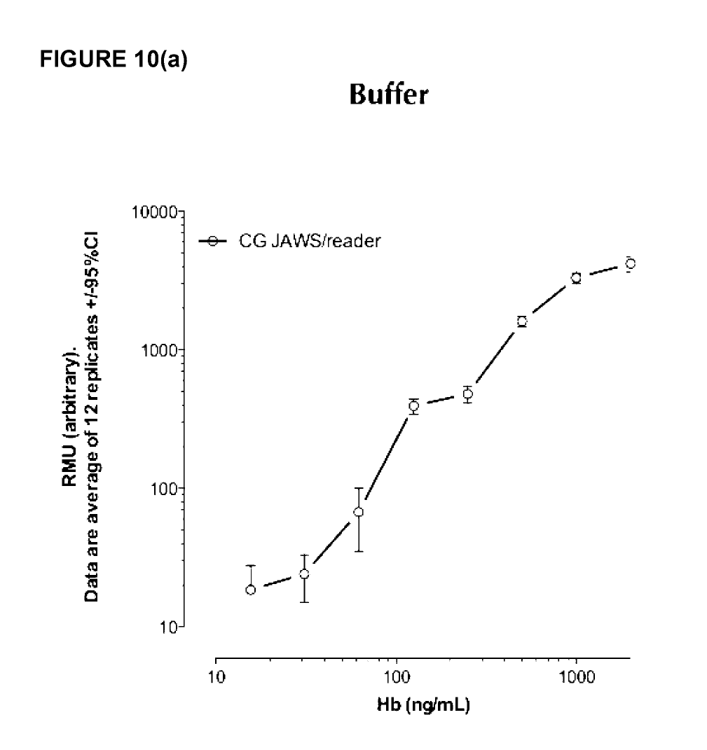

FIG. 9 shows the visual indication of a test device 1 being read by a reader that scans the visual indication of the test device 1 and provides a graphical output as shown in FIG. 9(b). This graphical output can then provide a quantitative test value based on the visual indication.

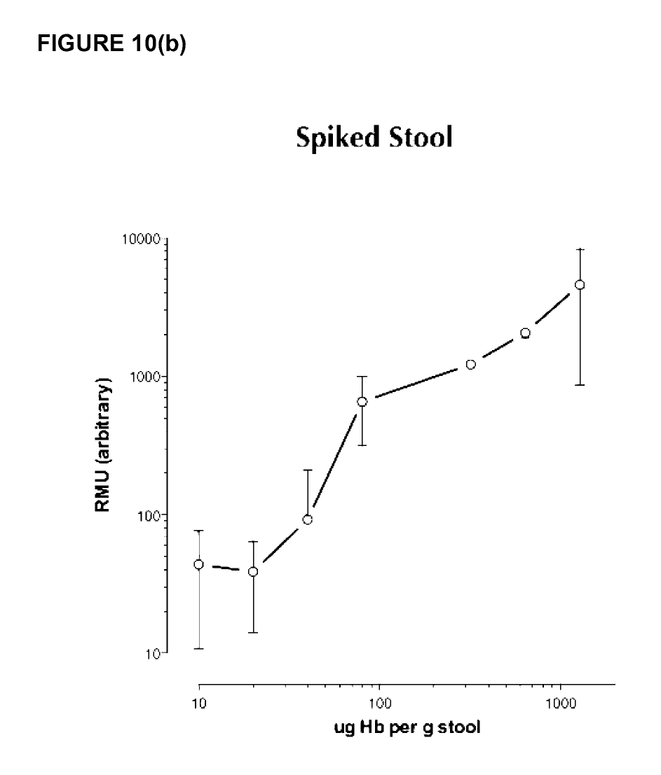

FIGS. 10(a) and 10(b) are respectively graphs showing a graphical representation of dose-response curves of the buffer system and stool system. Human haemoglobin was diluted at indicated concentrations and applied to the sample receiving port. Cards were read using a digital reader and signal intensities of control line and test line were expressed as ratio metric units.

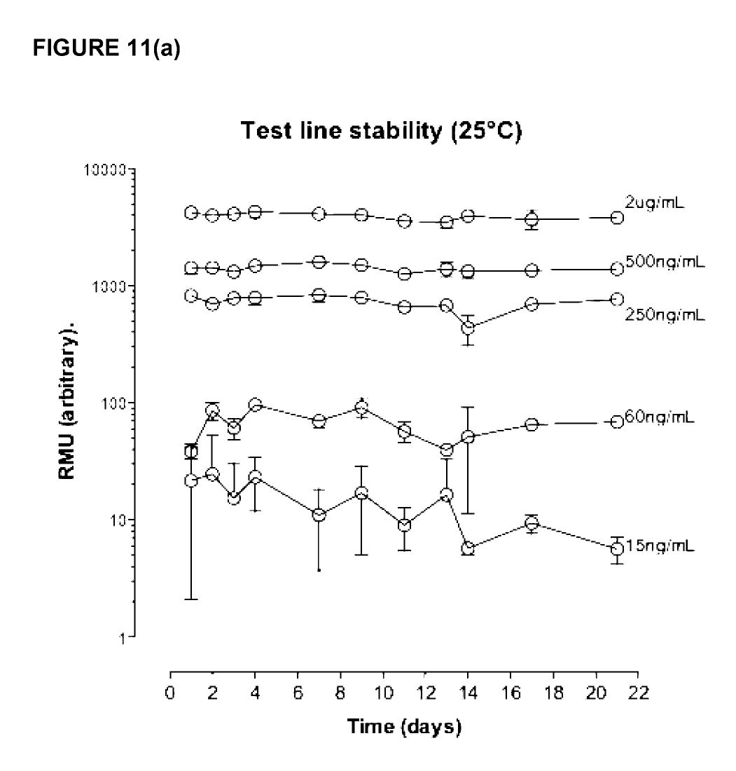

FIGS. 11(a) and 11(b) show the stability of the test result after storage of the device at 25° C. and 40° C. Human haemoglobin was diluted at indicated concentrations and applied to the sample application port. Cards were read using a digital reader and signal intensities of control line and test line were expressed as ratio metric units. Cards were stored at 25 degrees Celsius for 21 days and read at indicated intervals.

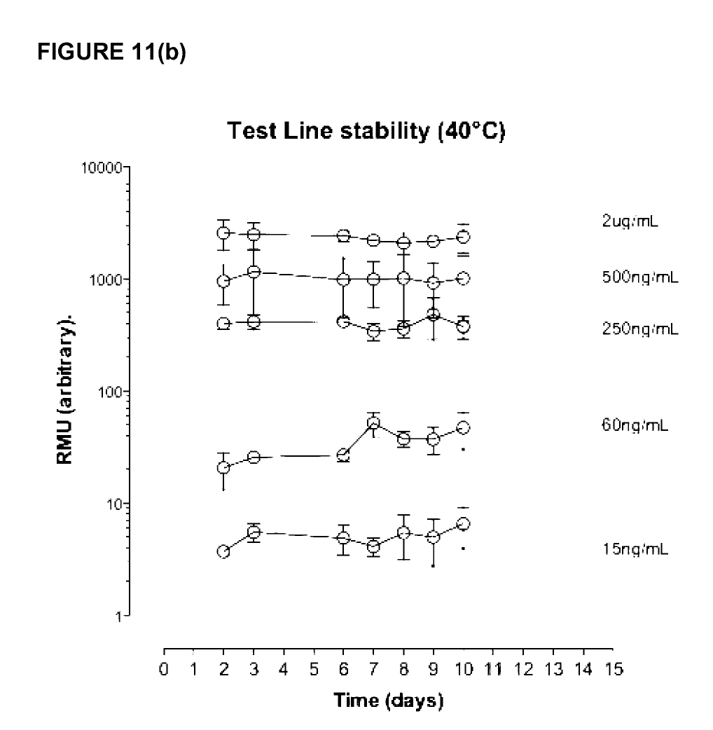

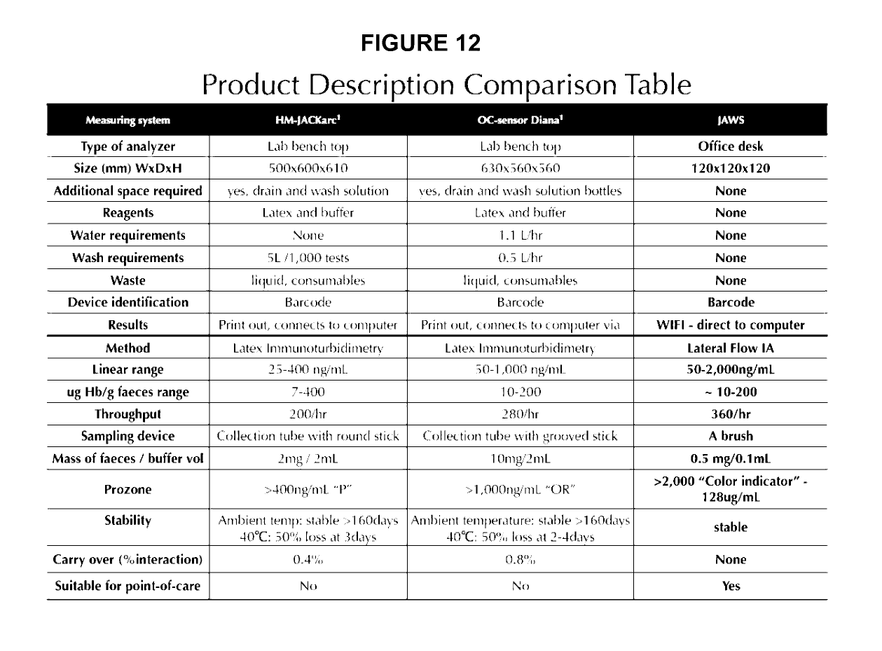

FIG. 12 is a table comparing the requirements and methods used with conventional FOBT and FIT with the test requirements and methods used for the test device 1 according to the present invention.

The test device 1 according to the present invention therefore allows for the immediate testing of collected samples on site without the need to use the facilities of an off site pathology lab. This self-testing aspect of the test device also makes it suitable for use in the home or in the field where no laboratory facilities are available.

While the present invention has been described with respect to its use in FOBTs and FITs, it is to be appreciated that the present invention can be used in other applications such as the sampling and analysis of other biological fluids such as blood, urine, semen and saliva, or may be adapted to analyse the presence of contaminants in ground water, or bacteria such as E. coli in food.

Persons skilled in the art will recognise that many modifications or variations may be made to the test device described in detail herein in order to suit other testing purposes or by way of adaption for optimal function, without departing from the spirit and scope of the present invention as broadly described above.

1. A method for testing an analyte in a collected sample using a test device including an enclosure having at least one sample receiving port for receiving the collected sample; a test strip located within the enclosure, the test strip containing at least one reagent for detecting the analyte and for providing an indication showing a test result for the collected sample, the enclosure including a detection arrangement for allowing detection of the indication of the test strip; and a sample receiving matrix positioned behind the at least one sample receiving port and having a defined saturation capacity, the sample receiving matrix being impregnated with reagents for pre-treatment of the sample and being in liquid-conductive communication with the test strip, said method comprising: delivering one or more samples to the at least one sample receiving port to saturate the sample receiving matrix such that a quantified amount of the sample is transferred to the test strip and an indication of the test result is effected.

2-36. (canceled)

37. A test device for testing an analyte in a collected sample comprising: an enclosure having at least one sample receiving port for receiving the collected sample; a test strip located within the enclosure, the test strip containing at least one reagent for detecting the analyte and for providing an indication showing a test result for the collected sample, the enclosure including a detection arrangement for allowing detection of the indication of the test strip; a sample receiving matrix positioned behind the at least one sample receiving port and having a defined saturation capacity, the sample receiving matrix being impregnated with reagents for pre-treatment of the sample and being in liquid-conductive communication with the test strip, wherein one or more said samples can be delivered to the at least one sample receiving port to saturate the sample receiving matrix such that a quantified amount of the sample is transferred to the test strip and an indication of the test result is effected.

38. A test device according to claim 37, wherein said indication is a visual indication, said detection arrangement is a viewing arrangement and said detection is viewing.

39. A test device according to claim 37, wherein said indication is non-visual and is detected using spectrometry, fluorimetry and magnetometry.

40. A test device according to claim 37, further comprising a sample collection apparatus for collecting and delivering the one or more samples to the at least one sample receiving port.

41. A test device according to claim 40, wherein the sample collection apparatus is a brush or a brush-like apparatus having flexible or semi-flexible bristles, wherein the volume of the sample collected by the brush or brush like apparatus is less than, equal to or greater than the defined saturation capacity of the sample receiving matrix.

42. A test device according to claim 37, further comprising an obscuring arrangement for hiding at least the indication.

43. A test device according to claim 42, wherein the obscuring arrangement comprises a hinged flap locatable over the means for covering and hiding the indication, and for allowing subsequent of the indication.

44. A test device according to claim 42 [[or 43]], the obscuring arrangement comprising a hinged flap locatable over the at least one sample receiving port for covering said at least one sample receiving port.

45. A test device according to claim 44, further comprising an identifier means for sealing the flap over the at least one sample receiving port after sample collection.

46. A test device according to claim 37, wherein the detection arrangement for allowing detection of the indication comprises at least one port located in the enclosure over the test strip.

47. A test device according to claim 37, wherein the enclosure further comprises an inspection port for observing a visual indication showing the flow of the sample through the test strip.

48. A test device according to claim 37, wherein the test strip is formed from an elongate strip of hydrophilic material, one end of the test strip providing a first location for sample collection, and opposing end of the test strip providing a second location for the indication showing the test result.

49. A test device according to claim 37, wherein the sample receiving matrix is formed of hydrophilic material.

50. A test device according to claim 37, wherein the sample receiving matrix is dimensioned to provide a said defined saturation capacity for receiving one or more said samples.

51. A test device according to claim 37, wherein the sample receiving matrix is dimensioned to provide a said defined saturation capacity equal to the volume of the sample required to complete the test of the device to thereby standardise the amount of the sample used in the test.

52. A test device according to claim 37, wherein the sample receiving matrix provides filtration of solids from the collected sample before transfer to the test strip.

53. A test device according to claim 37, wherein the sample receiving matrix contains at least one reagent for solubilising and buffering the analyte in the collected sample before transfer to the test strip.

54. A test device according to claim 37, wherein the sample receiving matrix contains one or more lytic agents for lysing and releasing the contents of any cells present in the collected sample.

55. A test device according to claim 37, wherein the sample receiving matrix contains one or more surfactants to prevent non-specific binding and loss of the analyte to the matrix and the test strip.

56. A test device according to claim 37, wherein the reagent for detecting the analyte is provided in the sample receiving matrix.

57. A test device according to claim 37, wherein the enclosure is formed of a sheet material and including a front panel wherein is located at the at least one sample receiving port, and the means for allowing viewing of the visual indication of the test strip, and a rear panel for supporting the test strip, the test strip being located between said front and rear panels.

58. A test device according to claim 57, wherein the sheet material is plastic.

60. A test device according to claim 57, wherein the sheet material is waterproof cardboard.

61. A test device according to claim 60 wherein said reagent for detecting haemoglobin is anti-globin antibody conjugated to a visualisation means.

62. A test device according to claim 61 wherein said anti-globin antibody is impregnated either within the sample receiving matrix or downstream from said sample receiving matrix.

63. A test device according to claim 37 wherein said test strip additionally includes a reagent for detecting haem.

64. A test device according to claim 63 wherein said reagent for detecting haem is located at the distal end of the test strip, preferably beyond the immunodetection zone.

65. A test device according to claim 64 wherein said reagent for detecting haem is a peroxidase reagent and a chromogen.
